(19) United States
(12) Patent Application Publication
(10) Pub. No.: US 2021/0078017 A1
(43) Pub. Date: Mar. 18, 2021

(54) ENVIRONMENTALLY FRIENDLY FLOTATION AGENT

(71) Applicant: Biosepar Gesellschaft Fur Medizin- Und Labortechnik Mbh, Simbach (DE)

(72) Inventor: Andreas Szabados, Grunwald (DE)

(73) Assignee: BIOSEPAR GESELLSCHAFT FÜR MEDIZIN- UND LABORTECHNIK MBH, Simbach (DE)

(21) Appl. No.: 16/630,536

(22) PCT Filed: Jul. 12, 2018
(86) PCT No.: PCT/EP2018/069046

§ 371 Date: Jan. 13, 2020

(30) Foreign Application Priority Data
Jul. 13, 2017 (DE) 10 2017 115 736.0

(51) Int. Cl. B03D 1/018; B01D 21/00; G01N 1/28; G01N 33/53

(52) U.S. Cl. CPC B03D 1/018; B01D 21/0084; G01N 2333/43539; G01N 33/5308; G01N 1/28

(57) ABSTRACT

A stool sample evaluation flotation agent comprising at least 0.01 mol/kg of a first compound which, in a dissolved state, releases anions by a buffer effect to generate and stabilize a neutral pH environment having a pH between 6.0 and 8.0. A method of stool sample examination involving separating endoparasites from a stool sample, the method comprising mixing the stool sample with a flotation agent comprising at least 0.01 mol/kg of a first compound which, in a dissolved state, releases anions by a buffer effect to generate and stabilize a neutral pH environment having a pH between 6.0 and 8.0

### REFERENCE TO RELATED APPLICATIONS

This application is a U.S. national stage application based on PCT/EP2018/069046, filed Jul. 12, 2018, claiming priority from Germany application no. 10 2017 115 736.0, filed Jul. 13, 2017, the entire disclosures of which are incorporated herein by reference.

### FIELD OF THE INVENTION

The present invention relates to the use of a flotation agent used in a method of examination for the separation of microbes, in particular endoparasites, from a stool sample.

### BACKGROUND

Coproscopy deals with the examination of the feces of mainly mammals, especially domestic and farm animals, for microbes, in particular endoparasites. Reference books, such as “Die koproskopische Diagnostik von Endoparasiten in der Veterinärmedizin (Coprological diagnosis of endoparasites in veterinary medicine)”, Ronald Schämschke, 2013, describe the general practice in coprological examinations to diagnose endoparasites in veterinary medicine.

Various examination methods are used in the field of coproscopy, both for reasons of concentration of the microbes and for better visualisation. For instance, the sedimentation method and the flotation method are known, which can also be combined with each other. The corresponding methods are described in detail in Schämschke's reference book mentioned above.

According to Schämschke, saturated saline solutions, sodium nitrate solutions, zinc sulphate solutions, zinc chloride solutions, sodium chloride/zinc chloride solutions or sucrose solutions are frequently used as flotation agents, each of the aforementioned flotation agents having certain advantages or disadvantages.

The use of the saline solution has advantages primarily in terms of environmental friendliness, but the morphological characteristics of the microbes/parasites change very quickly, often already during diagnostic processing, which can make microscopic detection difficult or even impossible.

Solutions of sodium nitrate and zinc salts (heavy metal compound) are highly polluting substances that are potentially hazardous to health. They require special treatment and must be disposed of as hazardous waste at a higher cost. Furthermore, high procurement costs are associated with the use of the aforementioned chemicals.

Zinc salt compounds are also highly caustic and also acutely dangerous for the respiratory organs, skin, mucous membranes and eyes (cornea, lens, tear duct).

With sucrose solutions, solutions can only be prepared up to a specific gravity of 1.25 at the most. However, this is not sufficient to detect all forms of parasites. In addition, the rapid morphological changes, as in the case of sodium chloride (see above), and also the rapid formation of crystals on the microscope slide make microscopic diagnosis difficult.

### SUMMARY OF THE INVENTION

It is the object of the invention to provide a flotation agent with excellent separation properties and, in particular, an environmentally friendly compound for the isolation of microbes from a stool sample within the framework of an examination for microbes.

The present invention solves this problem by an inventive application with the features of claim 1.

Advantageous embodiments of the invention are the subject matter of the dependent claims.

### BRIEF DESCRIPTION OF THE FIGURE

FIG. 1 a block diagram of a method for carrying out a coproscopic examination of a feces sample or stool sample in accordance with the invention.

### DETAILED DESCRIPTION OF PREFERRED EMBODIMENTS

A flotation agent according to the invention is used for an examination method in which microbes, in particular endoparasites, are separated from a stool sample. The purpose of isolating the microbes is to ensure that further examination, e.g. microscopic observation, is not disturbed by matrix substances in the stool sample.

The examination method can generally be applied to a wide range of living organisms, including humans if necessary. The examination method is of primary importance as a so-called coproscopic examination, especially in veterinary medicine for the identification of so-called endoparasites.

According to one aspect of the invention, the new flotation agent can be used as a solid, in particular as a solid mixture in undissolved form, and commercially distributed. This solid should then be dissolved or suspended, preferably during the test, by adding a prescribed and/or recommended amount of solvent. The preferred molar proportions of individual components in the flotation agent in the solid are based on one kilogram of the flotation agent.

In accordance with a further aspect of the invention, the new flotation agent can, however, also be offered and marketed preferably as a liquid in the form of a solution or suspension, with a predefined specific weight of the flotation agent.

In this case, the preferred molar proportions with regard to the components refer to one litre of the flotation agent.

According to the invention, the new flotation agent comprises at least 0.01 mol/l or at least 0.01 mol/kg of a first compound which, when dissolved, i.e. partially dissolved or fully dissolved, releases anions, preferably organic anions.

Especially preferred is a concentration of the first compound of at least 1 mol/l or at least 1 mol/kg.

According to the invention, the compound serves to generate and stabilize a neutral pH-value. Such a neutral pH-value is preferably in the range between pH=6.0 and pH=8.0. The said compound forms a pH buffer system, preferably on dissociation, which buffers in the abovementioned neutral range.

However, the concentration of the compound of the flotation agent in a liquid, e.g. a solvent, can preferably be up to 6 mol/litre or per kg liquid.

Organic anions in the context of the present invention are, in particular, anions which are released during the dissociation of organic acids or their salts, such as acetate ions, fruit acids, in particular citric acid etc.

Further preferred embodiments of the invention are the subject matter of the dependent claims.

The flotation agent can advantageously contain a solvent. The preferred solvent for the application is water. In addition, a dihydric, trihydric or polyhydric alcohol and/or an ester of such an alcohol, preferably glycerol, can also be used advantageously to improve the solubility, particularly of the organic constituents in the feces. Of course, several of the said alcohols or esters can also be used, e.g. in an aqueous mixture.

The preferred concentration of the said alcohols and/or esters is 0.1 to 50% by weight based on the total weight of the flotation agent.

In the dissolved state, the first compound and/or a second compound of the flotation agent can release cations which also serve to generate and stabilise the abovementioned neutral pH range by a buffer effect. Therefore the compounds are salts. It is an advantage if the first and/or the second compound is a sodium salt. Sodium salts usually have a high solubility in protic solvents, such as water, so that a high concentration of these salts is soluble in the solvent.

In accordance with a further aspect of the invention, the first compound can be an acid, and the second compound can be an alkaline solution or a solid hydroxide, in particular sodium hydroxide. The acid can be neutralised by adding the alkaline solution to achieve a favourable pH value between 6 and 8.

It is also possible that the second compound is formed as sodium salt and is present in the flotation agent at a concentration of at least 0.01 mol/l.

The organic anions of the first compound may preferably be the anions of at least one fruit acid or its salts. Typical preferred fruit acids are citric acid, malic acid, gluconic acid, glycolic acid, benzoic acid, benzoic acid derivatives, tartaric acid, lactic acid, oxalic acid and/or salicylic acid.

Succinic acid and its salts can also be used.

It is particularly advantageous if the organic anions of the first compound are citrate ions.

The flotation agent may also contain an additive in the form of a detergent. Detergents are anionic and cationic surfactants and emulsifiers, e.g. soaps and/or fatty acids.

The detergent can preferably comprise at least a soap, in particular a potassium soap, or be in the form of a soap. In a preferably used detergent composition, the dishwashing detergent “Pril” from Henkel may be used.

The detergent may preferably be contained as an additive in a concentration of 0.001 to 1.5 mol/l or 0.001 to 1.5 mol/kg.

In addition, the flotation agent may contain at least 0.001 mol/l or at least 0.001 mol/kg of an additive which increases the density of water and thus also the specific weight of water, the solvent. In this way, the specific weight of the flotation agent can also be adjusted as necessary, depending on the stool sample.

The additive which increases the density of water may contain at least one mono- and/or disaccharide. A preferred disaccharide in the context of the present invention is sucrose. Alternatively or additionally, in addition to mono- and disaccharides, also their caramelised compounds, in particular fructose, galactose, glucose, maltose, xylitol and isomalt, can preferably be used as additives, which in the context of the present invention are to be understood as caramel components derived and/or produced from mono- and/or disaccharides.

In the case of caramel, the additive contains at least partially unreacted mono- and disaccharides and their products of polymers, cations and/or aldehydes formed during caramelisation.

The flotation agent may, in particular, be a suspension or solution. In this case, the specific weight of the flotation agent is already adjusted by matching it with the solvent.

The flotation agent is preferably free or substantially free of zinc ions and/or other heavy metals, in particular toxic and/or polluting heavy metals. In the context of the present invention, “substantially free” means a concentration of less than 0.01 mmol/l or mmol/kg of zinc ions in the flotation agent.

The flotation agent is preferably free or substantially free from phosphates, sulphates and/or nitrates. Also in this context, “substantially free” means a concentration of less than 0.01 mmol/l or mmol/kg.

The method of examination is, in particular, a coproscopic examination, especially in the veterinary and/or human medicine field.

The flotation agent can also be used for a combined flotation and sedimentation method within the framework of a coproscopic examination.

The above-described properties, features and advantages of this invention, as well as the manner in which they are achieved, will become clearer in connection with the following description of the embodiments, which are explained in more detail in connection with the drawing. The drawings show:

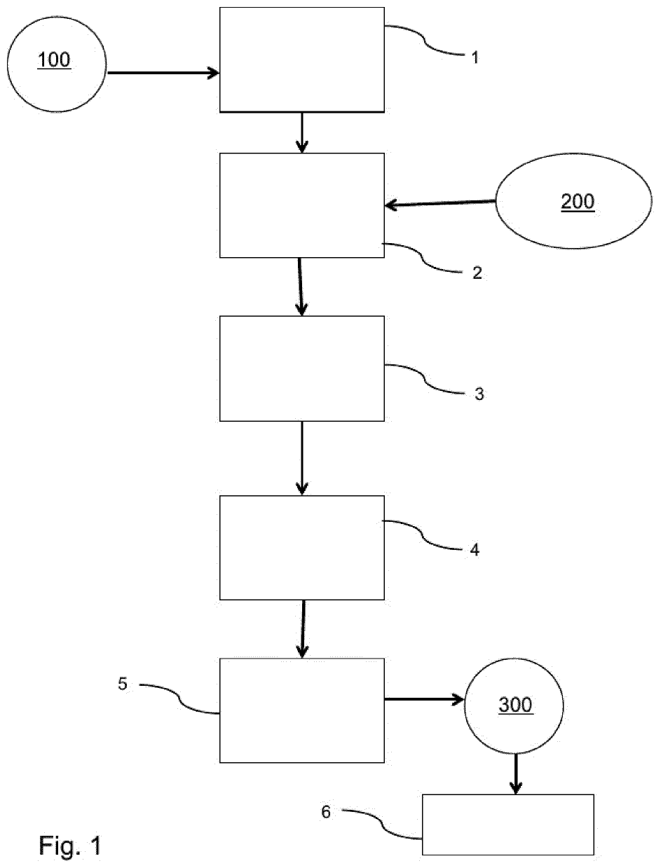

FIG. 1 a block diagram of a method for carrying out a coproscopic examination of a feces sample or stool sample 100 by means of the flotation agent used in accordance with the invention.

Coproscopic examinations make it possible to isolate so-called endoparasites from the feces of living beings and to examine them in various ways, e.g. microscopically.

Other faecal components can obstruct the visibility of the endoparasites. This is why different isolation variants are used in the coproscopic examination to separate the endoparasitic components of the feces from other components.

With regard to the isolation variants, the flotation method and the sedimentation method are known, among others. The flotation method is described in more detail below, but it can also be combined with the sedimentation method. Thus it is possible to first carry out a sedimentation method and then a flotation method, e.g. for the examination of horse feces. This means that the sensitivity of detection can be increased compared to the mere flotation method, so that all forms of parasites can be detected qualitatively and quantitatively with a higher hit rate.

In the flotation method, the addition of a flotation agent causes the endoparasitic components of the feces and, possibly, other microorganisms to float. Hereby, among others, cestoda and nematode eggs, i.e. eggs of tapeworms and nematodes, Cyclophyllidea, all protozoa, as well as e.g. coccidial oocysts and/or Giardia cysts and/or trophozoites can be detected.

First a stool sample 100 of a living being is provided (step 1). In order to avoid further interfering contaminations, e.g. soil contamination, during the later examination, the stool sample should be taken either at the rectum of the living being or immediately after defecation.

The stool sample 100 is then mixed with the flotation agent 200 (step 2). The flotation agent may be a solution or a suspension. The preferred solvent is water. However, it may be used as a mixture with a dihydric, trihydric or polyhydric alcohol and/or an ester of such an alcohol, preferably glycerol. It thus comprises at least a solvent, organic anions and inorganic and/or organic cations.

The organic anions originate from a compound which, on dissociation, releases these anions in the solution. Preferred organic anions in the context of the present invention are citrate ions. These can be dissolved by adding a citrate salt or by adding citric acid.

The ion concentration of the organic anions, in particular of the citrate ions in the solvent is preferably at least 0.01 mol/l, particularly preferably at least 1.0 mol/l, in particular 2.5 mol/l to 3.5 mol/l.

The inorganic or organic cations are preferably sodium ions. These can be added to the solvent in the form of sodium hydroxide and/or potassium hydroxide or a sodium and/or potassium salt.

The solvent is preferably water, in particular deionized water.

For example, in one version of the invention it is possible that the flotation agent consists only of sodium citrate and water.

However, the invention may also contain water, sodium hydroxide, potassium hydroxide and citric acid as well as other additives as applicable.

One of these additives can be at least a detergent. Such a detergent can influence the interfacial properties of the feces components so that, for example, better wetting can be achieved with the flotation agent. A preferred detergent within the scope of the present invention is a soap, preferably potassium soap.

Preferably, this can be achieved in a concentration of 0.001 to 1.5% by weight in the flotation agent.

A further additive is NaCl and/or KCl. This may preferably be contained in the flotation agent in a concentration of 0.5 to 5 mol/l or 0.5 to 5 mol/kg.

As a further additive, a liquid or a highly viscous mass can be added which at 20° C. has a specific weight which is at least 35% higher than that of water. Such a liquid can, for example, be a sugar-containing solution. Preferably the sugar solution has a concentration of sugar of at least 0.001 mol/kg, preferably at least 1.2 mol/kg in water, preferably 1.3 to 4.0 mol/kg. A disaccharide, such as sucrose, can preferably be used as a sugar.

A caramel-containing and/or cane sugar-containing solution should also be understood as a sugar-containing solution. Liquid caramel can also be used as an alternative to a sugar solution, preferably to adjust the specific weight. Preferably, the concentration and type of sugar in the sugar-containing solution and/or caramel should be selected in such a way that subsequent crystallisation on the slide does not occur for as long as possible, e.g. over 24 h.

Flotation agent 200 is preferably free of zinc, phosphate, sulphate or nitrate ions, or it contains these ions in a concentration of less than 0.01 mmol/kg.

The pH value of the flotation agent can preferably be in a range between pH=6 and pH=8.

The degree of saturation of one or more components of the flotation agent, for example sodium citrate or one of the said additives, can almost be reached in the flotation agent. Typically, the degree of saturation is given in grams per litre. “Almost reached” means that the concentration of the respective component per litre of flotation agent is at least 80%, based on the degree of saturation at 20° C. in the respective solvent, especially in water.

With reference to FIG. 1, in the second step 2 the stool sample 100 is mixed with the flotation agent 200 described above. The S:F ratio is preferably between 1:8 and 1:12, producing a faecal suspension.

The specific weight of the flotation agent for producing the faecal suspension should preferably be between 1.30 and 1.32. Accordingly, the flotation agent can be adjusted to the specific weight by adding the citrate source, the carbohydrate source and, if necessary, the additives, preferably NaCl and/or KCl and the detergent. The density can be determined using a flexural resonator or a hydrometer.

Then, in a third step 3, the above suspension is sieved to separate coarse components. A sieve with a predefined mesh size can be used for this purpose.

Optionally, further flotation agent 200 can be added for washing or for setting a measuring volume.

Subsequently, in a fourth step 4, there is a resting phase of 15-35 minutes. An extended resting period can allow a sinking of floating, surplus faecal matter that interferes with the examination. At the same time, the target microbes/parasites are optimally concentrated in the uppermost layer of the flotation agent.

As an alternative to the resting phase, centrifugal processing can also be used to achieve a better separation between floating feces components and the feces components that settle or dissolve and interfere with the examination.

The floating feces now contain the microbes 300 to be examined, such as larvae, eggs of tapeworms and threadworms, cysts, oocysts etc.

In a fifth step 5, these can be taken from the suspension with a so-called drop collector in the form of a wire eyelet or a fine pipette, deposited on a microscope slide, and used for further examination in step 6, e.g. for microscopic observation.

The use of the said flotation agent makes it possible, in particular, to feed the faecal suspension into the sewage system after the examination. There is no need for separate disposal due to substances that pollute the environment or are harmful to it.

The flotation agent comprising the abovementioned ions and additives also has excellent solubility in water. The viscosity increases after the addition of water only to an extent that does not affect the flotation of the microbes and thus the optimal result of the examination.

The flotation agent has been described above as a solution or suspension with water as a solvent. However, the flotation agent can also be formed exclusively from solids or from a viscous mass, which is further dissolved and diluted by the addition of further solvent.

All in all, the use of the said flotation agent represents an environmentally friendly and environmentally sound alternative with a very efficient isolation of parasites compared to previous flotation agents.

All in all, the use of the said flotation agent represents an environmentally friendly and environmentally sound alternative compared to previous flotation agents, while at the same time providing very efficient isolation of parasites without impairing their morphology.

### LIST OF REFERENCE SIGNS

1 step 1
2 step 2
3 step 3
4 step 4
5 step 5
6 step 6
100 stool sample
200 flotation agent
300 microbe sample

1-15. (canceled)

16. A method of stool sample examination involving separating endoparasites from a stool sample, the method comprising mixing the stool sample with a flotation agent comprising at least 0.01 mol/kg of a first compound which, in a dissolved state, releases anions by a buffer effect to generate and stabilise a neutral pH environment having a pH between 6.0 and 8.0.

17. The method of claim 16 wherein the flotation agent comprises water and one or more di-, tri- and/or higher-value alcohols and/or their esters.

18. The method of claim 17 wherein the di-, tri- and/or higher-value alcohols and/or esters thereof are present in the flotation agent in a concentration of 0.1% to 50% by weight

19. The method of claim 16 wherein the first compound and/or a second compound of the flotation agent, when dissolved, releases cations as a result of a buffer effect to generate and stabilise a neutral pH environment.

20. The method of claim 19 wherein the first and/or the second compound is a sodium salt or potassium salt.

21. The method of claim 20 wherein the second compound is formed as the sodium salt and is present in the flotation agent in a concentration of at least 0.01 mol/l.

22. The method of claim 16 wherein the anions of the first compound are organic anions of at least one fruit acid or a salt of a fruit acid and/or are formed as citrate ions.

23. The method of claim 16 wherein the flotation agent comprises an additive which is a detergent.

24. The method of claim 23 wherein the detergent is contained in the flotation agent (200) in a concentration of 0.001 to 1.5 mol/kg.

25. The method of claim 16 wherein the flotation agent comprises at least 0.001 mol/kg of an additive which increases the density of water.

26. The method of claim 25 wherein the additive which increases the density of water comprises a mono- and/or disaccharide, comprises sucrose or consists of sucrose, or comprises caramel constituents derived and/or prepared from one or more mono- and/or disaccharides, or consists of the caramel constituents.

27. The method of claim 16 wherein the flotation agent is a suspension or solution.

28. The method of claim 16 wherein the flotation agent is substantially free of zinc ions, heavy metal ions, phosphates, sulphates, and nitrates.

29. The method of claim 16 wherein the examination is a coproscopic examination in the field of human and veterinary medicine.

30. The method of claim 16 wherein the stool sample examination comprises a combined flotation and sedimentation method.

31. The method of claim 16 wherein the flotation agent comprises:
water;
from 0.1 to 50 wt % of one or more di-, tri- and/or higher-value alcohols and/or their esters;
at least 0.01 mol/l of a sodium salt or potassium salt which releases cations, wherein the sodium salt or potassium salt is either a component of the first compound, or is a second compound distinct from the first compound;
from 0.001 to 1.5 mol/kg of a detergent; and
at least 0.001 mol/l of an additive which increases density of water;
wherein the flotation agent is free of zinc ions, heavy metal ions, phosphates, sulphates, and nitrates; and
the anions of the first compound are organic anions of at least one fruit acid or a salt of a fruit acid, and/or are formed as citrate ions.
